SEQUENCE LISTING (9 sequences)

SEQ ID NO: 1 - protein, 15 aa, Artificial Sequence
  Heteroconjugate construct
MDMCSAGWLADRSVR

SEQ ID NO: 2 - protein, 15 aa, Artificial Sequence
  Heteroconjugate construct
  Xaa (12)..(12): Xaa is Citrulline
MDMCSAGWLADXSVR

SEQ ID NO: 3 - protein, 15 aa, Artificial Sequence
  Heteroconjugate construct
DGQEEKAGVVSTGLI

SEQ ID NO: 4 - protein, 33 aa, Artificial Sequence
  Heteroconjugate construct
  Xaa (30)..(30): Xaa is Citrulline
DGQEEKAGVVSTGLIGGGMDMCSAGWLADXSVR

SEQ ID NO: 5 - protein, 33 aa, Artificial Sequence
  Heteroconjugate construct
DGQEEKAGVVSTGLIGGGMDMCSAGWLADRSVR

SEQ ID NO: 6 - protein, 9 aa, Artificial Sequence
  Heteroconjugate construct
VQGEESNDK

SEQ ID NO: 7 - protein, 13 aa, Artificial Sequence
  Heteroconjugate construct
DLLKNGERIEKVE

SEQ ID NO: 8 - protein, 15 aa, Artificial Sequence
  Heteroconjugate construct
ATEGRVRVNSAYQDK

SEQ ID NO: 9 - protein, 15 aa, Artificial Sequence
  Heteroconjugate construct
  Xaa (5)..(5): Xaa is Citrulline
ATEGXVRVNSAYQDK

(19) United States
(12) Patent Application Publication
(10) Pub. No.: US 2022/0218806 A1
(43) Pub. Date: Jul. 14, 2022

(54) PEPTIDES AND CONJUGATES FOR TREATMENT OF ARTHRITIS

(71) Applicant: CRL-SCI Corporation, Vienna, VA (US)

(72) Inventors: Daniel H. Zimmerman, Bethesda, MD (US); Tibor T Glant, Chicago, IL (US); Katalin MIKECZ, Chicago, IL (US); Roy E Carambula, Owing Mills, MD (US)

(73) Assignee: CRL-SCI Corporation, Vienna, VA (US)

(21) Appl. No.: 17/610,319

(22) PCT Filed: May 11, 2019
(86) PCT No.: PCT/US2020/032376

§ 371 Date: Nov. 10, 2021

Related U.S. Application Data
(60) Provisional application No. 62/846,609, filed on May 11, 2019.

(51) Int. Cl. A61K 39/00; A61K 47/64; A61K 39/39; A61P 19/02

(52) U.S. Cl. CPC A61K 39/0008; A61K 47/646; A61K 39/39; A61K 2039/55577; A61K 2039/55566; A61K 2039/55555; A61K 2039/55572; A61P 19/02

(57) ABSTRACT

The invention relates to peptide heteroconjugates useful in treating Rheumatoid Arthritis (RA). The peptide heteroconjugates include a portion of the Human aggrecan protein conjugated to an Immune Cell Binding Ligand (ICBL) by a direct bond or divalent linker. The peptide heteroconjugates including combinations thereof, can be used as a vaccination for RA when combined with an adjuvant and administered to a subject.

### REFERENCE TO SEQUENCE LISTING

This application contains a “Sequence Listing” submitted as an electronic .txt file named “CS-138PCTUS_SEQ.txt.” The subject matter of the “Sequence Listing” is incorporated herein by reference.

### FIELD OF INVENTION

The invention relates to peptide heteroconjugates useful in treating Rheumatoid Arthritis (RA). The peptide heteroconjugates include a portion of the Human aggrecan protein conjugated to an Immune Cell Binding Ligand (ICBL) by a direct bond or divalent linker. The peptide heteroconjugates including combinations thereof, can be used as a vaccination for RA when combined with an adjuvant and administered to a subject.

### BACKGROUND

Autoimmune diseases are driven by the cytokines produced by T cells, B cells, macrophages, dendritic cells and other cells. The cytokines can define different T cell responses. Rheumatoid Arthritis (RA) is an autoimmune disorder characterized by systemic inflammation and progressive joint deterioration. Autoimmune diseases, such as RA, occur when antibodies produced by B cells mount an immune response against self antigens. According to the CDC, an estimated 1.5 million people in the US had RA in 2007, with an estimated 50 million worldwide having some form of arthritis. In 2012, three of the ten highest selling drugs in the US were RA biologics for treatment of symptoms or ablative treatment of activated cells or proinflammatory cytokines.

Vaccines protect against disease by stimulating the production of antibodies against a causative agent of the disease. Peptide vaccines can include useful peptides that can be incorporated into an immunogen and can have the ability to generate an appropriate immune response. There is a need for peptide vaccines that can be used to treat or vaccinate against RA. Ligand Epitope Antigen Presentation System (LEAPS) vaccines could be modulatory rather than ablative therapy. LEAPS is a novel immunization technology that enables the design and synthesis of relatively small peptide immunogens and determination of the resultant immune response. Further, determination of the T cell response driving the disease could allow personalized medicine for RA, with the choice of LEAPS vaccine determined by the T cell response and antigens involved.

Generating an effective immune response imposes certain requirements because not all immune responses are effective. Indeed, immune responses can be harmful, and some antigens can evoke or elicit antibodies when an immune response of a cellular nature is needed. Autoimmune arthritis imposes additional requirements since it is often a self-epitope. As such, a peptide epitope may sometimes need to be incorporated or linked to another peptide forming another larger component. The larger combination of peptides can then impose further restrictions because the components must be compatible and not interfere with one another. The known peptides include P49 which contains a key site for citrullination at arginine 305 (R₃₀₅) that appears to be important in autoimmune arthritis in animal models and in man (Markovics, A., Ocskó, T., Katz, R. S., Buzás, E. I., Glant, T. T., & Mikecz, K. (2016), Immune Recognition of Citrullinated Proteoglycan Aggrecan Epitopes in Mice with Proteoglycan-Induced Arthritis and in Patients with Rheumatoid Arthritis. PloS One, 11(7), e0160284). However, epitope P49 has several undesirable features for commercial applications based on lower reactivity as seen in adjacent peptides when examined in pools for various responses (See also WO/2008/043157 (“Thomas”)). Similar studies of the same region albeit using a different starting position nomenclature of 280-292 for the same sequence and shifted away from the amino terminus of P49 to the carboxyl end just before the W₃₀₁ in or W₂₈₂ eliminating the two M and C support the same conclusion (Aggarwal, A., Srivastava, R., & Agrawal, S. (2013). T cell responses to citrullinated self-peptides in patients with Rheumatoid Arthritis. Rheumatology International, 33(9), 2359-63). Moreover, the known literature notes that an adjacent R₂₈₅R₂₈₆ did not contain D (Markovics, A., Ocskó, T., Katz, R. S., Buzás, E. I., Glant, T. T., & Mikecz, K. (2016) Immune Recognition of Citrullinated Proteoglycan Aggrecan Epitopes in Mice with Proteoglycan-Induced Arthritis and in Patients with Rheumatoid Arthritis. PloS One, 11(7), e0160284).

There is a need to provide a composition and methods for delivering a therapeutic goal in autoimmune inflammatory diseases such as Rheumatoid Arthritis to suppress responses against (auto)antigens present in one or more target organ. The need includes providing an antigen-specific therapy. The need further includes compositions and methods for having improved reactivity. The need can further include combination therapies and delivery of one more compositions to improve therapy. For increased efficacy of treatment, there is a need for multi-epitope vaccines incorporating distinct epitopes that are located in distant regions of the PG molecule involved in arthritis induction.

### BRIEF DESCRIPTION OF THE DRAWINGS

FIG. 1 shows an overview of a method for evaluating responses to the described peptide heteroconjugates.

FIG. 2 shows a model showing several domains and the relationship of PG70 and PG275 epitopes in the G1 domain.

FIG. 3 shows an arthritis visual score time course of development after immunization.

FIG. 4 shows representative histopathology stains of diseased mice ankles after immunization.

FIG. 5 shows representative histopathology stains of diseased mice hind paws after immunization.

FIG. 6 shows sera antibody specificity expression for each vaccinated group of mice.

FIG. 7 shows expression of intracellular cytokines and FoxP3 in spleen T cells.

FIG. 8 shows a net amount of secreted cytokines by vaccinated spleen cells.

### BRIEF SUMMARY

The first aspect of the invention is related to peptide heteroconjugates having a formula P₁-x-P₂ or P₂-x-P₁; wherein P₁ is selected from the group consisting of SEQ ID NO.'s 3 and 7; wherein P₂ is selected from the group consisting of SEQ ID NO.'s 1 and 2; and wherein x is a direct bond or divalent linker for covalently bonding P₁ and P₂.

In any embodiment, P₁ can be SEQ ID NO. 3.

In any embodiment, P₂ can be SEQ ID NO. 2.

In any embodiment, x can be a divalent linker comprising a sequence GGG.

The second aspect of the invention is related to a composition comprising at least one peptide heteroconjugate having a formula P₁-x-P₂ or P₂-x-P₁; wherein P₁ is selected from the group consisting of SEQ ID NO.'s 3 and 7; wherein P₂ is selected from the group consisting of SEQ ID NO.'s 1 and 2; wherein x is a direct bond or divalent linker for covalently bonding P₁ and P₂; and further comprising an adjuvant.

In any embodiment, the composition can further comprise a second peptide heteroconjugate.

In any embodiment, the second peptide heteroconjugate can have a formula P₃-x-P₄ or P₄-x-P₃; wherein P₁ is selected from the group consisting of SEQ ID NO.'s 3 and 7; P₃ is selected from the group consisting of SEQ ID NO.'s 3 and 7; wherein P₄ is selected from the group consisting of SEQ ID NO.'s 8 and 9; and wherein x is a direct bond or divalent linker for covalently bonding P₃ and P₄.

In any embodiment, the second peptide heteroconjugate can be from PG, collagen, vimentin, and may include citrullination of arginine and/or glycosylation of other residues such as serine or threonine.

In any embodiment, P₁ can be SEQ ID NO. 3.

In any embodiment, P₂ can be SEQ ID NO. 2.

In any embodiment, x can be a divalent linker comprising a sequence GGG.

In any embodiment, the adjuvant can be selected from the group consisting of Seppic ISA51vg, Freund's incomplete adjuvant, Lipid A, MPL, AS01, AS03, AS04, Novasomes and Liposomes, MF59, QS21, IS01, IS03, IS04, or combinations thereof.

The third aspect of the invention is related to a method of treating a subject. In any embodiment, the method can comprise the steps of administering the composition of the second aspect of the invention to the subject.

In any embodiment, the composition can further comprise a second peptide heteroconjugate.

In any embodiment, the second peptide heteroconjugate can have a formula P₃-x-P₄ or P₄-x-P₃; wherein P₁ is selected from the group consisting of SEQ ID NO.'s 3 and 7; P₃ is selected from the group consisting of SEQ ID NO.'s 3 and 7; wherein P₄ is selected from the group consisting of SEQ ID NO.'s 8 and 9; and wherein x is a direct bond or divalent linker for covalently bonding P₃ and P₄.

In any embodiment, the second peptide heteroconjugate can be from PG, collagen, vimentin, and may include citrullination of arginine and/or glycosylation of other residues such as serine or threonine.

In any embodiment, P₁ can be SEQ ID NO. 3.

In any embodiment, P₂ can be SEQ ID NO. 2.

In any embodiment, x can be a divalent linker comprising a sequence GGG.

In any embodiment, the adjuvant can be selected from the group consisting of Seppic ISA51vg, Freund's incomplete adjuvant, Lipid A, MPL, AS01, AS03, AS04, Novasomes and Liposomes, MF59, QS21, IS01, IS03, IS04, or combinations thereof.

In any embodiment, the subject can be human.

In any embodiment, the composition can be administered to the subject multiple times.

In any embodiment, the method can comprise administering between 1 mg and 50 mg of the peptide heteroconjugate each of the multiple times.

In any embodiment, a time period between each administration of the composition can be between 1 week and 1 year, between 1 week and 3 weeks, between 2 weeks and 3 weeks, between 3 weeks and 6 months, between 1 month and 6 months, between 3 months and 6 months, between 1 month and 1 year, or between 6 months and 1 year.

In any embodiment, the subject can be a mouse.

In any embodiment, the method can comprise administering between 0.05 μg and 0.1 μg, between 0.1 μg and 10 μg, between 10 μg and 100 μg, or between 100 μg and 300 μg of the peptide heteroconjugate.

In any embodiment, the subject can be a rat.

In any embodiment, the method can comprise administering between 0.05 μg and 3 mg of the peptide heteroconjugate.

### DETAILED DESCRIPTION

Unless defined otherwise, all technical and scientific terms used herein generally have the same meaning as commonly understood by one of ordinary skill in the relevant art.

The articles “a” and “an” are used herein to refer to one or to more than one (i.e., to at least one) of the grammatical object of the article. By way of example, “an element” means one element or more than one element.

The trademarked term “L.E.A.P.S.” (also referred to as “L.E.A.P.S.” or LEAPS) refers to “Ligand Epitope Antigen Presentation System” and stands for a peptide based antigen delivery technology for directing immune responses toward a desired outcome.

The term “adjuvant” refers to substance that accelerates, prolongs or enhances antigen-specific immune responses when used in combination with vaccine antigens.

The terms “administering,” “administer,” “delivering,” “deliver,” “introducing,” and “introduce” can be used interchangeably to indicate the introduction of a therapeutic or diagnostic agent into the body of a patient in need thereof to treat a disease or condition, and can further mean the introduction of any agent into the body for any purpose.

The term “citrulline” indicates an α-amino acid. The name is derived from citrullus. Citrulline has the idealized chemical formula H₂NC(O)NH(CH₂)₃CH(NH₂)CO₂H, and is a key intermediate in the urea cycle, the pathway by which mammals excrete ammonia. Citrulline is made from ornithine and carbamoyl phosphate in one of the central reactions in the urea cycle. It is also produced from arginine as a by-product of the reaction catalyzed by NOS family (NOS; EC 1.14.13.39). Arginine is first oxidized into N-hydroxyl-arginine, which is then further oxidized to citrulline concomitant with release of nitric oxide. Citrulline is formed in the protein, after protein synthesis, by enzymatic or chemical conversion of arginine to citrulline. The in-situ process is catalyzed by one or more variants (1-5) of the enzyme referred to PAD for peptidyl-dinimido-amino peptide.

The term “comprising” includes the recited steps, elements, structures or compositions of matter and does not exclude any un-recited elements, structures or compositions of matter.

The term “consisting of” includes and is limited to whatever follows the phrase the phrase “consisting of.” Thus, the phrase indicates that the limited elements are required or mandatory and that no other elements may be present.

The phrase “consisting essentially of” includes any elements listed after the phrase and is limited to other elements that do not interfere with or contribute to the activity or action specified in the disclosure for the listed elements. Thus, the phrase indicates that the listed elements are required or mandatory but that other elements are optional and may or may not be present, depending upon whether or not they affect the activity or action of the listed elements.

The term “effective amount” is an amount of a therapeutic which produces a therapeutic response, including an immune response, in the subject to which the therapeutic is administered.

The terms “conjugate,” “conjugation” and similar terms refer to two species being spatially associated with each other by covalent linkage, non-covalent binding or by a combination of covalent linkage and non-covalent binding. For example, an antibody can be conjugated to an epitope through non-covalent binding to the epitope as well as the antibody serving to conjugate the epitope (such as a cell surface marker) to a compound that is linked to the antibody.

The term “divalent linker” refers to any moiety having a structure forming a peptide bond to a first peptide moiety and forming a second bond to a second peptide moiety.

A “heteroconjugate” refers to a protein or peptide containing at least two amino acid sequences covalently linked to form a single molecule, wherein two sequences originate or are homologous to proteins expressed by different genes.

The terms “peptide” and “peptide construct” broadly refer to any molecule or part of a molecule including two or more amino acid residues linked by a peptide bond. The term “peptide construct” can also include molecular species where only part of the molecule has peptide character and/or where two parts of the molecular species formed of peptide bonds are covalently linked.

The term “subject” or “patient” refers to an animal, including mice and humans, to which a therapeutic agent is administered.

The terms “treating” and “treatment,” as related to treating or treatment of immune cells, refers to bringing an immune cell into contact with a substance or composition for a time period sufficient to cause a change in phenotype.

The term “vaccine” refers to a composition containing one or more antigens that stimulate an immune response when administered to vertebrates in vivo.

### Heteroconjugates

Table 1 shows peptides of one of the major proteins (aggrecan or Proteoglycan) thought to be involved as epitopes and antigens in RA and animal models from several publications including the Thomas R. patent applications. PCT/AU2007/001555 and PCT/AU2013/000303.

TABLE I
Select region of Human aggrecan protein with region and peptides
studied by others identified

```
    210 220 230 240 250
AGWLADQTVR YPIHTPREGC YGDKDEFPGV RTYGIRDTNE TYDVYCFAEE
                                                                        (294/275)
                                                                          -P49---
                                                                                        -P50-
                                            ---P48----------- -P51
    260 270 280 290 300
MEGEVFY ATS PEKFTFQEAA NECRRLGA [symbol] L ATTGHVYLAW QAGMDMCSAG
                                                                            291 to
-P49----
-P50--------
-P51----------
  -P52------------
      310 320 330 340 350
WLAD [symbol] SV [symbol] YP ISKA [symbol] PNCGG NLLGVRTVYV HANQTGYPDP SSRYDAICYT
306
```

The novel peptides of this invention include peptide heteroconjugates having the following formulae (I) or (II):

P₁-x-P₂ (I)

P₂-x-P₁ (II)

where P₁ is an immune cell binding ligand (ICBL), P₂ is either PG275 or PG275Cit, and x is a direct bond or divalent linker for covalently bonding P₁ and P₂. Attachment of an ICBL to a T cell epitope containing peptide determines the resultant response.

A key consideration in developing an immunization is to preserve the key position of the R₃₀₅ known to be involved in binding to both antibodies and T cells based on the effects of substitutions with citrulline which enhances the efficacy of the peptide and to position the R₃₀₅ in an appropriate location and maintain the local proximity of net charge and hydrophobicity around this “305” site.

One concern regarding peptide vaccines is the use of only a limited number of epitopes because an agent responsible for a disease process can oftentimes involve multiple epitopes. Another concern is a possibility of escape such as due to genetic drift if only a single epitope is used. However, several factors related to the criteria used in the epitope selection process can mitigate the concerns. Moreover, combinations of peptides vaccines and/or peptide vaccines containing multiple epitopes can confer high efficacy. In particular, combinations of peptides vaccines and/or peptide vaccines containing multiple epitopes may be of a benefit if one epitope is missing or altered. The effective use of numerous therapeutic monoclonal antibodies supports the use of combinations of peptides vaccines and/or peptide vaccines containing multiple epitopes insofar as monoclonal antibodies by definition have a single epitope specificity. The likelihood of both epitopes being altered or missing is generally lower, hence supporting a concept of combinations of peptides vaccines and/or peptide vaccines containing multiple epitopes. Further, two epitopes distant from one another can minimize the likelihood of a genetic missing element. That not all epitopes induce the same type of Th2/Treg protective response and use of two epitopes can also minimize the probability that both may be missing.

Based on the concerns, two core analogues of P₂₉₁ are identified:

PG275
SEQ ID NO. 1
MDMCSAGWLADRSVR
PG275Cit
SEQ ID NO. 2
MDMCSAGWLAD(Cit)SVR

The two core analogues, SEQ ID NO. 1 and SEQ ID NO. 2, can be conjugated with an immune cell binding ligand (ICBL) to promote the immunogenicity of an epitope. Examples of ICBLs include:

DerG
SEQ ID NO. 3
DGQEEKAGVVSTGLIGGG-amide
J
SEQ ID NO. 7
DLLKNGERIEKVEGGG-amide

Peptide J is derived from human beta-2-microglobulin. J-LEAPS vaccines activate mouse and human precursors to differentiate into dendritic cells (DCs) that produce IL12p70 (Rosenthal, K S, and Zimmerman, D H. J-LEAPS vaccines elicit antigen specific Th1 responses by promoting maturation of type 1 dendritic cells (DC1). 2017 AIMS Allergy and Immunology 1(2): 89-100). J-LEAPS conjugate vaccines or J-LEAPS induced DCs have been shown to be protective in HSV-1, Influenza A, Murine Her2Neu cancer, EAM and CIA challenge mode (Zimmerman, et al., 2010, Int Immunopharmacol. 2010 10(4):412-21; Rosenthal et al., 2017, J Immunol Res. 2017; 2017:361; Rosenthal K S, Taylor P, Zimmerman D H. J-LEAPS peptide and LEAPS dendritic cell vaccines. Microb Biotechnol. 2012 March; 5(2):203-13; Taylor P R, Koski G K, Paustian C C, Bailey E, Cohen P A, Moore F B, Zimmerman D H, Rosenthal K S. J-LEAPS vaccines initiate murine Th1 responses by activating dendritic cells. Vaccine. 2010 Aug. 2; 28(34):5533-42; Taylor P R, Paustian C C, Koski G K, Zimmerman D H, Rosenthal K S. Maturation of dendritic cell precursors into IL12-producing DCs by J-LEAPS immunogens. Cell Immunol. 2010; 262(1):1-5; Zimmerman D H, Taylor P, Bendele A, Carambula R, Duzant Y, Lowe V, O'Neill S P, Talor E, Rosenthal K S. CEL-2000: A therapeutic vaccine for rheumatoid arthritis arrests disease development and alters serum cytokine/chemokine patterns in the bovine collagen type II induced arthritis in the DBA mouse model. Int Immunopharmacol. 2010 April; 10(4):412-21; and Cihakova D, Barin J G, Baldeviano G C, Kimura M, Talor M V, Zimmerman D H, Talor E, Rose N R. L.E.A.P.S. heteroconjugate is able to prevent and treat experimental autoimmune myocarditis by altering trafficking of autoaggressive cells to the heart. Int Immunopharmacol. 2008 May; 8(5):624-33).

Peptide Der-G is derived from the beta chain of human MHC II. Der-G-LEAPS vaccines induce immune responses with Th2 associated IgG isotypes and cytokine profiles. The DerG-LEAPS vaccines are not protective or therapeutic in the HSV, influenza, EAM and CIA models. DerG-LEAPS vaccine is therapeutic for the PGIA and GIA RA models. Proteoglycan-induced arthritis and recombinant human proteoglycan aggrecan G1 domain-induced arthritis in BALB/c mice resemble two subtypes of Rheumatoid Arthritis. Arthritis and Rheumatism, 63(5), 1312-21). The conjugation of the core analogues of P₂₉₁ with the ICBLs give the peptide heteroconjugates having the formulae P₁-x-P₂ or P₂-x-P₁. One non-limiting example, SEQ ID NO. 3 is conjugated with SEQ ID NO. 2 to give the peptide heteroconjugate:

SEQ ID NO. 4
DerG-PG275Cit
GQEEKAGVVSTGLIGGGMDMCSAGWLAD(Cit)SVR

Alternatively, SEQ ID NO. 3 can be conjugated with SEQ ID NO. 1 to give the peptide heteroconjugate:

DerG-PG275
SEQ ID NO. 5
DGQEEKAGVVSTGLIGGGMDMCSAGWLADSVR

As described, peptide J can be used as the ICBL an conjugated to SEQ ID NO. 1 or SEQ ID NO. 2. Table 2 shows the peptide sequences used, along with two peptides derived from PG70, which may be combined with the PG275 peptides for vaccination, as described:

TABLE 2

| SEQ ID | Common | |
|---|---|---|
| NO. | name | Peptide sequence |
| 1 | PG275 | MDMCSAGWLADRSVR-amide |
| 2 | PG275Cit | MDMCSAGWLADCitSVR-amide |
| 3 | DerG | DGQEEKAGVVSTGLIGGG-amide |
| 4 | DerG- | DGQEEKAGVVSTGLIGGGMDMCSA |
| | PG275Cit | GWLADCitSVR-amide |
| 5 | DerG- | GQEEKAGVVSTGLIGGGMDMCSAG |
| | PG275 | WLADRSVR-amide |
| 6 | E | VQGEESNDKGGG-amide |
| 7 | J | DLLKNGERIEKVEGGG-amide |
| 8 | PG70 | ATEGRVRVNSAYQDK-amide |
| 9 | PG70Cit | ATEG(Cit)VRVNSAYQDK-amide |

To treat or immunize subjects against RA, the described peptide heteroconjugates can be administered to the subject in an adjuvant. Any adjuvant capable of enhancing antigen-specific immune responses in combination with the described peptide heteroconjugates can be used. Non-limiting examples of adjuvants can include Seppic ISA51vg, Freund's incomplete adjuvant, Lipid A, MPL, AS01, AS03, AS04, Novasomes and Liposomes, MF59, QS21, IS01, IS03, IS04, or combinations thereof.

The amount of the peptide heteroconjugate administered to the subject can vary with size, age, and species of the subject. In human subjects, between 1 mg and 50 mg of the peptide heteroconjugate can be administered per dose. Generally, it is preferable with human subjects to spread out the doses. For example, the peptide heteroconjugate can be administered to the subject every week, every 2 weeks, every three weeks, or longer. In any embodiment, the time period between each administration can be is between 1 week and 1 year, between 1 week and 3 weeks, between 2 weeks and 3 weeks, between 3 weeks and 6 months, between 1 month and 6 months, between 3 months and 6 months, between 1 month and 1 year, between 6 months and 1 year, or longer than 1 year between administrations.

For administration to mice, between 0.05 μg and 300 μg of the peptide heteroconjugate can be administered, including between 0.05 μg and 0.1 μg, between 0.1 μg and 10 μg, between 10 μg and 100 μg, or between 100 μg and 300 μg of the peptide heteroconjugate. In rats, between 0.05 μg and 3 mg of the peptide heteroconjugate can be administered.

The vaccines may be administered with an adjuvant on a regular regimen by any route, including intradermal, intramuscular, subcutaneous or as a cutaneous transdermal or nasal delivery.

In certain embodiments, a combination vaccine may be administered. The combination vaccine can include the peptide heteroconjugates using PG275 and PG275Cit in combination with a peptide heteroconjugate using PG70. Previously, A DerG LEAPS conjugate of PG70 (Cel-4000) was shown to be therapeutic via up modulation of Th2 and Treg, i.e. up modulation of IL4, IL10 and CD4+CD25+ FoxP3 cells (Mikecz et al. 2017 Vaccine 35:4048-4056). A multi-epitope combination vaccine can have certain advantages over a single epitope peptide vaccine. A multi-epitope vaccine may cover the different epitopes involved in the disease process of a pathogen or autoimmune antigen. However, based on the single epitope action of many monoclonal antibodies, single epitope activity may be effective. A multi-epitope vaccine may still be effective in an individual with responses to a different epitope or an altered epitope, especially if the other epitope is presented together or in the same context. With a single epitope vaccine, if the epitope selected is missing in the antigen in the affected individual or animal the vaccine may be ineffective. These concerns may be substantially overcome if a two epitope vaccine is used especially if the two epitopes are not closely linked or in proximity to one another and even more so if a three epitope vaccine with a third conjugate is used. Another advantage of a two epitope approach is in a possible event that one pathway is missing in the T cell recognition of antigen or the repertoire of immune response having a second redundant pathway or recognition. PG70 and PG275 incorporate distinct epitopes that are located in distant regions of the PG molecule involved in arthritis induction.

There are certain concerns with multi-epitope vaccines as well. The response to a different epitope may compete or interfere with that against the first conjugate. Further, one epitope could initiate an inappropriate response. However, neither of these concerns is found for the PG70 and PG275 vaccines. As described, the two epitopes appeared to act independently to induce different but therapeutic, and probably based on involvement of DerG binding to CD4, similar Th2/Treg, responses. DerG-PG70 induced antibodies against the epitope and DerG components. DerG-PG275Cit induced antibodies against neither the DerG element nor the PG275Cit element. The cytokine response elicited in vitro and the ICS response upon antigenic stimulation were different for each epitope and yet both appear to act via the Th2/Treg pathway based on the DerG affinity for CD4+ T cells.

### Methods

To study the efficacy of the described peptide heteroconjugates, LEAPS therapy in the cartilage proteoglycan (PG) induced arthritis (PGIA) model of RA using these new peptides and conjugates were examined Studies for efficacy were determined in a challenge disease model of human RA. The animal model of RA is induced by immunization of aging female BALB/c mice with human cartilage proteoglycan. The animal model is well documented and resembles human disease with spondyloarthropathy, the presence of RF and ACPA, and propensity for occurrence in older females (von Delwig, A., Locke, J., Robinson, J. H., & Ng, W.-F. (2010)). Response of Th17 cells to a citrullinated arteriogenic aggrecan peptide in patients with Rheumatoid Arthritis. Arthritis and Rheumatism, 62(1), 143-9). Immunization with PG or the G1 domain (containing most arthritogenic/dominant epitopes of human cartilage) induces PGIA or GIA disease (Glant, T. T., Radacs, M., Nagyeri, G., Olasz, K., Laszlo, A., Boldizsar, F., Mikecz, K. (2011). Proteoglycan-induced arthritis and recombinant human proteoglycan aggrecan G1 domain-induced arthritis in BALB/c mice resemble two subtypes of Rheumatoid Arthritis. Arthritis and Rheumatism, 63(5), 1312-21 and Glant et al. 2004 Autoimmunity: Methods and Protocols 102:313). PGIA and GIA disease are characterized by predominant Th1 IFNg cytokine production. Retired breeder female BALB/c mice received 3 intraperitoneal injections of 40 μg rhG1 in DDA adjuvant 3 weeks apart. Once the mice in the study reached a desired mean disease (GIA) score of 3 for the intended groups, the mice were randomized and assigned to the treatment groups and were vaccinated on days 0 and 14 subcutaneously with one of four treatments. The first group received ISA51vg adjuvant emulsified with PBS (adjuvant control); the second group received CEL-4000 in adjuvant, the third group received CEL-4000 and DerG-PG275Cit in adjuvant, and the fourth group received DerG-PG275Cit in adjuvant. The vaccines were formulated by one investigator and given under code to a second investigator immunizing the animals.

After the onset of disease, mice were divided in several (as indicated) separate equally size groups, each with a similar mean arthritis index (AI). One group was vaccinated with adjuvant only, and the other groups with one of two LEAPS conjugates of a PG epitope 70 (J-PG70 or DerG-PG70) in adjuvant or both DerG-PG70 AND DerG-PG275Cit. The same vaccination was repeated two weeks later. AI were measured by visual scoring of each limb as done before (Mikecz et al. 2017 and Zimmerman et al. 2010) every other day for a total of about 5 weeks since the start of therapeutic immunization. Upon euthanization of the mice in the groups A, C, D and E, the proportions of Th1, Th2, Th17, and regulatory T cell (Tregs) in the spleen cells were determined by flow cytometry. as well as these cells were examined for cytokine secretion, in addition for all Groups (A-E) sera collected and examined for cytokines and antibodies to the vaccine and limbs process for histological examination.

Epitope PG275 was synthesized as a citrullinated to form PG275Cit and conjugated to DerG to form DerG-PG275Cit. Citrullination is a common posttranslational modification to RA antigens. PG275cit occurs in 27% of RA patients. CEL-4000 and other peptides were supplied with free amino terminus and amidated C-terminus as a lyophilized acetate salt at >90% purity (RP-HPLC and MS+/−2amu). Neutravidin Microplates were purchased from Thermo Fisher and Biotin labeled peptides of DerG-PG70 (CEL-4000), PG70, DerG, DerG-PG275Cit, PG275Cit and Ova as a specificity control from Biomatik, prepared from dry peptide, dissolved, stored frozen, diluted and loaded onto plates at mg/mL just before conducting an immuno-assay. Each conjugate vaccine was evaluated alone and in combination. Multiple parameters were evaluated, as described.

To determine a visual arthritis score (VAS), swelling and redness of the paws were scored visually (scores: 0-4/paw, 0-16/mouse). One of skill in the art will understand that the terms “visual arthritis score (VAS),” visual score (VA),” and “visual arthritis index (VAI)” can be used interchangeably. Mice were sorted into 5 groups, each of a similar mean VAS (day 0). The scoring was done 3 times a week by two separate investigators independently of each other. Data was analyzed by a third investigator that determined if the two scoring investigators agreed. If the two investigators disagreed by more than 0.5 in animal's score, the scores were sent back for another scoring before analysis could proceed.

Animals were euthanized on day 35 after the first control or LEAPS vaccination, and sera was collected. For histopathology, hind limbs (from 5 mice/treatment groups plus 3 normal mice limbs) were dissected and fixed in 10% buffered formalin. The specimens were embedded in paraffin and sectioned. Adjacent tissue sections were stained separately with TB and HE. Microscopic analysis and scoring of joint damage were carried out independently under code by Bolder BioPath. Serum anti-LEAPS peptide antibody levels were determined by ELISA using Neutavidin plates and Biotin labeled peptides for loading onto plates. To determine cytokine production by spleen cell cultures, after a 4-day stimulation of spleen cells with 7.5 μg/ml rhG1 (or no treatment as control), cytokines secreted into the culture media were assayed by MagPix as 9-plex or 6-plex kits from R&D Systems. For intracellular cytokines and FoxP3 detection, spleen cells from 5 mice from each group were cultured with rhG1 for 4 days. The cells were treated with PMA, ionomycin, and GolgiStop for 4 hours, stained for cell surface markers and intracellular cytokines or Foxp3. Samples were run on a FACS Canto flow cytometer and the results analyzed by FACS Diva software. Statistical analysis of data was done using GraphPad Prism 7 software package. Nonparametric and multiple comparison test were conducted as described.

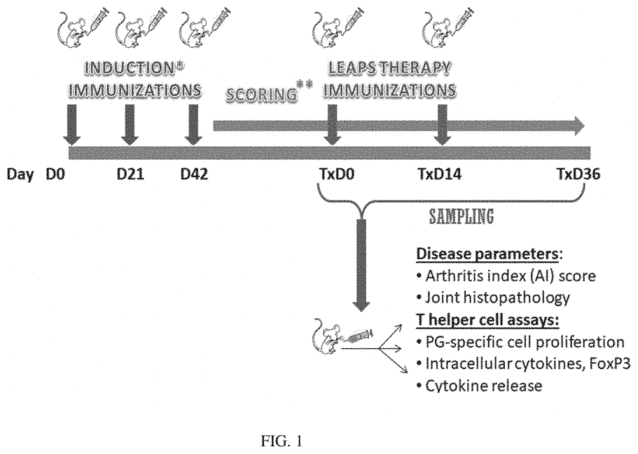

FIG. 1 shows an overview of a method for evaluating immunization of proteoglycan [PG] induced arthritis (PGIA) or recombinant huG1 domain (GIA), autoimmune models of Rheumatoid Arthritis with LEAPS therapy. On days DO, D21, and D42 mice are treated with an induction immunization. The inducing antigen can be human PG (hPG) or recombinant G1 domain of hPG (rhG1). The major immunogenic epitope is PG70-84. Thereafter, an arthritis score is determined for the mice. The mice are monitored from day 42, the day of the third induction immunization, until the arthritis score is greater than 3. The mice are grouped so that all groups have the same mean group score and range, and LEAPS immunization therapy is initiated on therapy day 0 (TxD0) and the arthritis scored every other day. Therapy immunization is also administered 14 days later, on day TxD14. Sampling is continued until day TxD36. The disease parameters used are the arthritis index score and a joint histopathology. T helper cell assays are also conducted, measuring DerG-PG70, PG70, DerG, PG275Cit, DerG-PG275Cit and, as an additional specificity control, Ova peptide-specific cell proliferation, intracellular cytokines, FoxP3, cytokine release, and serum anti-peptide antibodies.

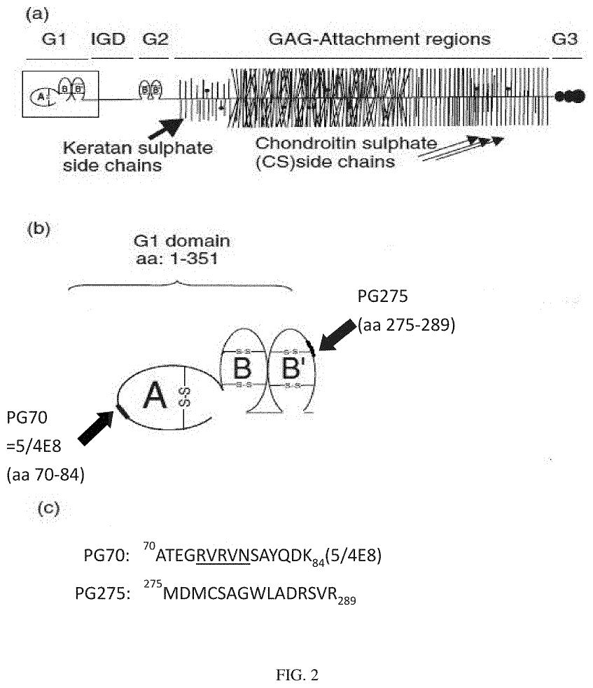

FIG. 2 shows a model showing several domains and the relationship of PG70 and PG275 epitopes in the G1 domain. The PG70 epitope is more immunogenic than PG70Cit in mice. The PG275Cit epitope is more immunogenic than PG275 in both mice and humans. PG275Cit can occur in 27% of RA patients.

In addition to studying PG275 and PG275Cit, PG70 and PG70Cit were also studied. PG70 is defined as proteoglycan aggrecan (G1) residues 70-84, and has the following sequence:

### SEQ ID NO. 8 PG70 ATEGRVRVNSAYQDK-amide

The citrulline version of PG70, called PG70Cit was also studied and has the following sequence:

### SEQ ID NO. 9 PG70CIT ATEG(Cit)VRVNSAYQDK-amide

In any heteroconjugate of the invention, one non-limiting embodiment of the linker can be a divalent linker between the ICBL and the epitope. In another non-limiting embodiment, the divalent linker can be a triple glycine linker. In other embodiments, the linker of the invention can be a direct link together in any order (i.e., N-terminal of one to C-terminal of other or vice versa). The peptide of the invention can be covalently bonded by a spacer or linker molecule by any method or composition known to those of skill in the art. With regard to linkers between the two domains, suitable examples include a thioether bond between an amino terminus bromoacetylated peptide and a carboxyl terminus cysteine, often preceded by a diglycine sequence (Zimmerman et al., supra), carbodiimide linkages, a multiple glycine, e.g., from 3 to 6 glycines, such as triglycine, with or without one or two serines, separation between the two entities, e.g., GGGS (SEQ ID NO. 7), GGGSS (SEQ ID NO. 8), GGGGS (SEQ ID NO. 9), GGGGSS (SEQ ID NO. 10), GGGSGGGS (SEQ ID NO. 11), etc., and other conventional linkages, such as, for example, the direct linkages such as, EDS, SPDP, and MBS, as disclosed in the aforementioned U.S. Pat. No. 5,652,342.

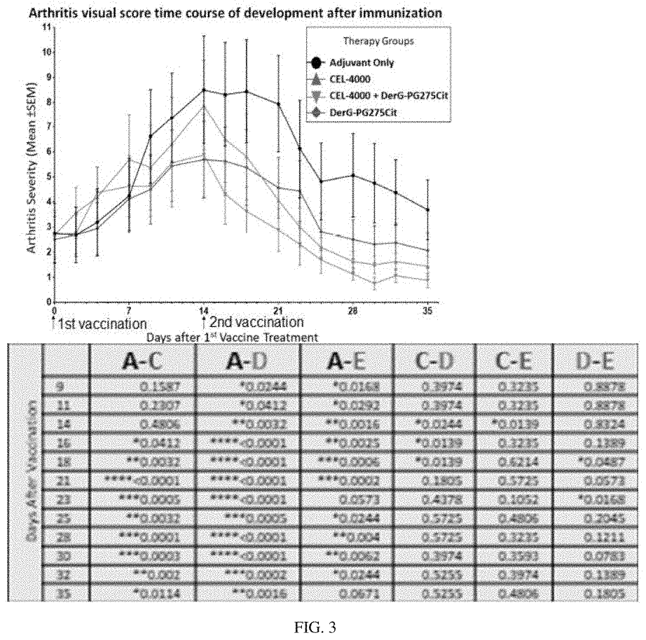

FIG. 3 shows an arthritis visual score time course of development after immunization. The mice were separated into four therapy groups: a control group that only received an adjuvant, a first group that received immunization with CEL-4000 in adjuvant, which is DerG-PG70, a third group that received a combination of CEL-4000 and DerGPG275Cit in adjuvant, and a fourth group that received solely DerGPG275Cit in adjuvant. The first vaccination was given to the mice on day 0 in the graph in FIG. 3, with a second vaccination on day 14. Arthritis severity was assessed by visual scoring every 3 days for 5 weeks. As illustrated in FIG. 3, Disease severity was significantly suppressed (reduced VAS) in mice treated with the LEAPS vaccines as compared with the adjuvant-treated control group. Surprisingly, the greatest suppression of disease severity was found in the group that received both CEL-4000 and DerGPG275Cit in adjuvant, suggesting that CEL-4000 and DerGPG275Cit may suppress the disease by different T helper 2/regulatory T cell (Th2/Treg)-associated protective mechanisms. In addition, CEL-4000 and DerGPG275Cit incorporate distinct epitopes that are located in distant regions of the PG molecule involved in arthritis induction. Further, neither heteroconjugate seemed to inhibit the other's therapeutic effect. Thus, a combination vaccine containing both CEL-4000 (DerG-PG70) and DerG-PG275Cit, could offer advantages in case one epitope or another was missing in the disease inducing situation.

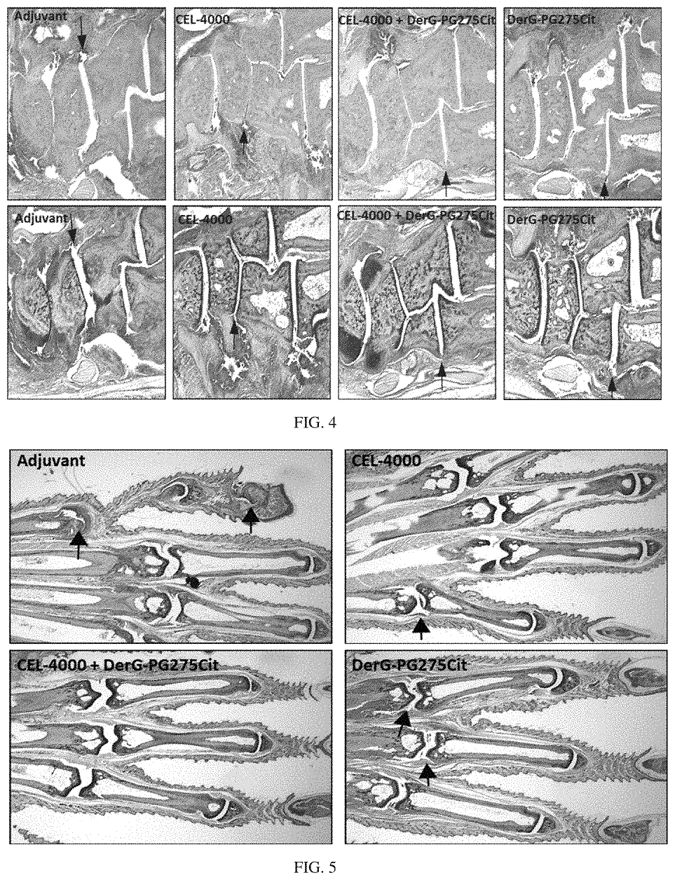

FIG. 4 shows representative histopathology stains of diseased mice ankles at 40× magnification for each of the four groups. The adjuvant only treated animals displayed marked inflammation and moderate cartilage damage with minimal pannus and bone resorption, as well as severe periosteal bone formation, in the ankle and several digit joints. The CEL-4000 treated animals displayed moderate inflammation and mild cartilage damage in the ankle and a single digit joint. The CEL-4000+DerG-PG275Cit animals displayed mild inflammation and cartilage damage with very minimal pannus and bone resorption, as well as moderate periosteal bone formation, in the ankle only. The DerG-PG275Cit animals displayed moderate inflammation and mild cartilage damage in the ankle and several digit joints.

FIG. 5 shows representative histopathology stains of diseased mice hind paws at 16× magnification for each of the four groups. The top left PBS in adjuvant animals displayed marked inflammation and moderate cartilage damage with minimal pannus and bone resorption, as well as severe periosteal bone formation, in several digit joints. The top right CEL-4000 treated animals displayed moderate inflammation and mild cartilage damage in a single digit joint. The bottom left CEL-4000+DerG-PG275Cit treated animals displayed no inflammation or cartilage damage in digit joints. The bottom right DerG-PG275Cit treated animals displayed moderate inflammation and mild cartilage damage in several digit joints.

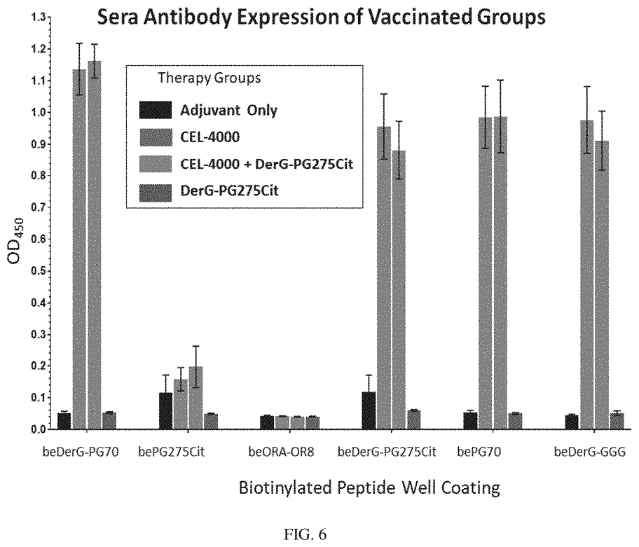

FIG. 6 shows sera antibody expression for each of the vaccinated groups. Serum antibodies from mice shown in FIG. 6 were assayed by biotinylated (be) peptide coating and NeutrAvidin ELISA. X-Axis. Peptide well coating (from left to right): beDerG-PG70, bePG275cit, beOVA-OR8, beDerG-PG275cit, bePG70, beDerG-GGG. Y-Axis: Optical Density 450 (OD) at 450 nm. The CEL-4000 and the Combination vaccine elicited antibodies but DerG-PG275cit did not.

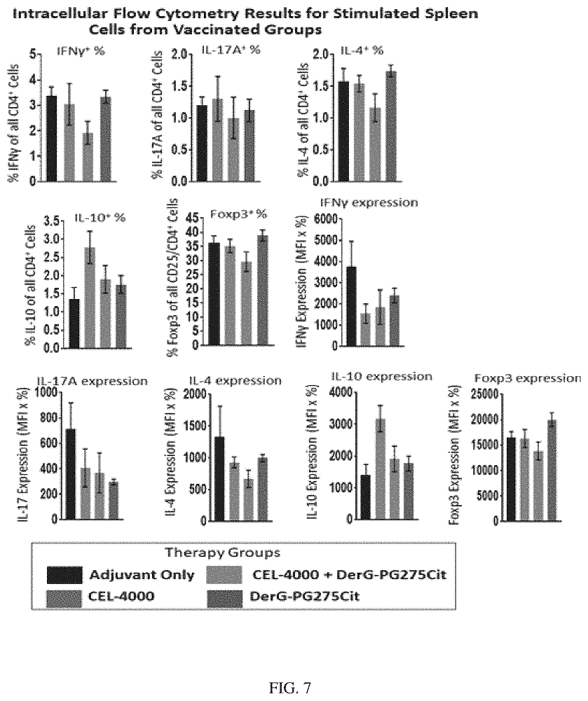

FIG. 7 shows expression of intracellular cytokines and FoxP3 in spleen T cells. The flow cytometry gating was set at CD4+ T cells (n=5 mice per group) Proinflammatory response Th1 is defined by IFNγ, TNFβ, IL2, and IL12, while proinflammatory response TH17 is defined by IL17, TNFα, IL22, and IL23. Anti-inflammatory and regulatory Th2 response is defined by IL4, IL5, and IL10, while regulatory Treg response is defined by TGFβ and IL10. A ratio of proinflammatory to regulatory or TH2 cytokines of greater than 1 indicates inflammatory and pre-treatment conditions. A ratio of proinflammatory to regulatory or TH2 cytokines of less than 1 indicates successful control of inflammatory conditions. All LEAPS-vaccinated mice produced slightly less Th1-associated cytokines, and cells from CEL-4000 treated group produced more Th2/Treg type IL10 than controls. The DerG-PG275Cit vaccine appeared to activate Treg cells.

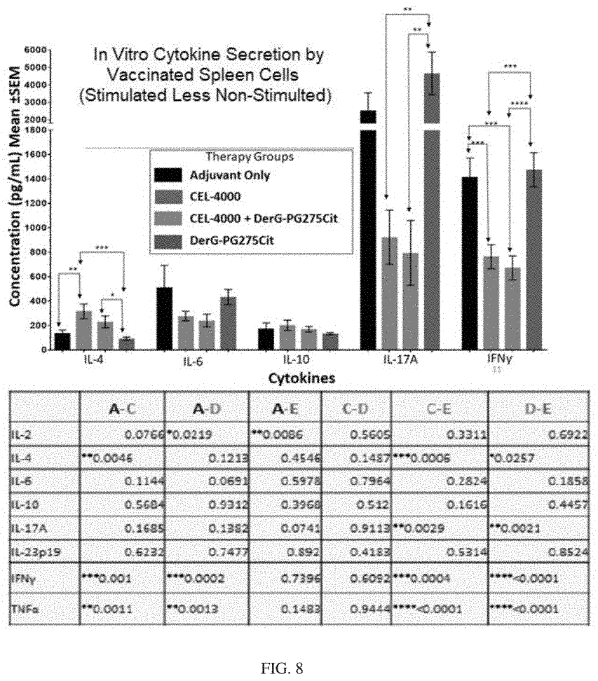

FIG. 8 shows the net (stimulated less non-stimulated cells) amounts of secreted cytokines by vaccinated spleen cells. Results from non-stimulated cells were subtracted from the results for stimulated cells. Outliers from IL-6 and IL-17A were identified and removed using Tietjen-Moore Test. CEL-4000 induced IL4 and suppressed IL6, IL17A and IFNγ. DerG-PG275cit induced IL17A and IFNγ but these cytokines were suppressed in the combined vaccine treated mice.

Based on the results, both DerG-PG70 and DerG-PG275Cit are therapeutic in the GIA model of RA with high efficacy, and may act by different mechanisms. DerG-PG275Cit does not induce antibodies to either DerG or PG275Cit or the conjugate. DerG-PG70 induces antibodies to itself that recognize DerG or PG70. DerG-PG70 appears to act by up modulation of Th2 and Treg cells and corresponding cytokines. DerG-PG275 does not appear to up modulate Th2 or Treg or Th1 cells or cytokines, but likely binds to CD4⁺ T cells. Thus, while either DerG-PG275 or DerG-PG70 can provide protection alone, a combination vaccine containing both DerG LEAPS conjugates could offer advantages in case one epitope or another was missing or mutated beyond recognition in the disease inducing situation or if the host did not recognize one epitope due to T cell repertoire.

In certain embodiments, other or additional conjugates can be included. For example, epitopes from PG, collagen, or vimentin can be used. The conjugates can include regular or post translational modifications, such as citrullination of arginine or glycosylation of other residues such as serine or threonine.

It will be apparent to one skilled in the art that various combinations and/or modifications and variations can be made in the dialysis system depending upon the specific needs for operation. Moreover, features illustrated or described as being part of an aspect of the invention can be included in the aspect of the invention, either alone or in combination

1. A peptide heteroconjugate having a formula:
P₁-x-P₂ or P₂-x-P₁; wherein
P₁ is selected from the group consisting of SEQ ID NO.'s 3 and 7; wherein P₂ is selected from the group consisting of SEQ ID NO.'s 1 and 2; and wherein x is a direct bond or divalent linker for covalently bonding P₁ and P₂.

2. The peptide heteroconjugate of claim 1, wherein P₁ is SEQ ID NO. 3.

3. The peptide heteroconjugate of claim 1, wherein P₂ is SEQ ID NO. 2.

4. The peptide heteroconjugate of claim 1, wherein x is a linker comprising a sequence GGG.

5. A composition comprising:
at least one peptide heteroconjugate having a formula
P₁-x-P₂ or P₂-x-P₁; wherein
P₁ is selected from the group consisting of SEQ ID NO.'s 3 and 7; wherein P₂ is selected from the group consisting of SEQ ID NO.'s 1 and 2; and wherein x is a direct bond or divalent linker for covalently bonding P₁ and P₂; and
an adjuvant.

6. The composition of claim 5, further comprising a second peptide heteroconjugate.

7. The composition of claim 6, the second peptide heteroconjugate having a formula
P₃-x-P₄ or P₄-x-P₃; wherein
P₃ is selected from the group consisting of SEQ ID NO.'s 3 and 7; wherein P₄ is SEQ ID NO. 8; and wherein x is a direct bond or divalent linker for covalently bonding P₃ and P₄.

8. The composition of claim 6, the second peptide heteroconjugate is from PG, collagen, vimentin, and may include citrullination of arginine and/or glycosylation of other residues such as serine or threonine.

9. The composition of claim 5, wherein P₁ is SEQ ID NO. 3.

10. The composition of claim 5, wherein P₂ is SEQ ID NO. 2.

11. The composition of claim 5, wherein x is a linker comprising a sequence GGG.

12. The composition of claim 5, wherein the adjuvant is selected from the group consisting of Seppic ISA51vg, Freund's incomplete adjuvant, Lipid A, MPL, AS01, AS03, AS04, QS21, Novasomes and Liposomes, MF59, QS21, IS01, IS03, IS04, or combinations thereof.

13. A method of treating a subject, comprising the steps of administering the composition of claim 5 to the subject.

14. The method of claim 13, wherein the composition further comprises a second peptide heteroconjugate.

15. The method of claim 14, wherein the second peptide heteroconjugate has a formula
P₃-x-P₄ or P₄-x-P₃; wherein
P₃ is selected from the group consisting of SEQ ID NO.'s 3 and 7; wherein P₄ is SEQ ID NO. 8 or SEQ ID NO. 9; and wherein x is a direct bond or divalent linker for covalently bonding P₃ and P₄.

16. The method of claim 14, wherein the second peptide heteroconjugate is from PG, collagen, vimentin, and may include citrullination of arginine and/or glycosylation of other residues such as serine or threonine.

17. The method of claim 13, wherein P₁ is SEQ ID NO. 3.

18. The method of claim 13, wherein P₂ is SEQ ID NO. 2.

19. The method of claim 13, wherein x is a linker comprising a sequence GGG.

20. The method of claim 13, wherein the adjuvant is selected from the group consisting of Seppic ISA51vg, Freund's incomplete adjuvant, Lipid A, MPL, AS01, AS03, AS04, QS21, Novasomes and Liposomes, MF59, QS21, IS01, IS03, IS04, or combinations thereof

21-28. (canceled)
